(19) United States
(12) Patent Application Publication
(10) Pub. No.: US 2026/0325716 A1
(43) Pub. Date: Oct. 22, 2026

(54) BONE AND CARTILAGE STEP BLADE MORSELIZER

(71) Applicants: Kevin R. Stone, San Francisco, CA (US); Mark Joseph, San Francisco, CA (US)

(72) Inventors: Kevin R. Stone, San Francisco, CA (US); Mark Joseph, San Francisco, CA (US)

(21) Appl. No.: 19/181,434

(22) Filed: Apr. 17, 2025

(51) Int. Cl. A61L 27/36

(52) U.S. Cl. CPC A61L 27/3691

(57) ABSTRACT

Disclosed is a surgical handheld rotary bone and articular cartilage morselizer that transforms bone segments, bone plugs, articular cartilage, and associated elements into uniform bits using a rotary cutting disc with stepped surfaces with recesses ending in an opening and a blade, and conical cutouts on cutting edges of the blade. Morselized bone/cartilage mix is combined with other ingredients and implanted into a bone or cartilage defect to aid repair of the defect.

### FIELD

The present disclosure generally relates to a surgical handheld rotary bone and articular cartilage step morselizer, (hereto referred to as the Device), that transforms bone segments, bone plugs, articular cartilage, bone marrow, and associated elements, (hereto referred to as the Elements), that have been immediately removed from a patient or mammal, and morselizes these elements into a fine Paste, (hereto referred to as Paste). This occurs in seconds, in a sterile operating room environment, the Paste to be immediately reinserted into the patient or mammal, (hereto referred to as the Patient), for the purpose of regenerating articular cartilage and/or bone.

### BACKGROUND

There are a number of surgical operating room procedures that are intended to repair articular cartilage lesions in joints, and to regrow bone lesions and fractures. These techniques involve using a sharp toothed trephine, (or hollow toothed drill with a push rod inside), to remove a cylindrical plug of articular cartilage and underlying bone from a Patient that range in size up to 10 mm in diameter and up to 20 mm long, down to 5 mm in diameter by 10 mm long. They are harvested in an area that will regrow and not be compromised. The plug is then processed into a fine Paste of morsels to immediately be impacted it into the prepared articular cartilage lesion. The Paste then integrates with the underlying bone and articular cartilage to grow new articular cartilage over time, thus repairing the lesion. Alternately, the Elements can be used as a filler to graft bone in orthopedic procedures. The Elements can also be integrated with additives to perform various functions. The key to success involves quickly making a consistent micro sized Paste in a manner that preserves a high degree of viable living cells needed for regeneration. This surgical process presents a number of challenges because the Elements and bone section tends to be extremely hard, dense and very difficult to cut or morselize.

There are a number of current methods to process the Elements into small bits to make a Paste. These may involve grinding, mashing, extruding, and chopping techniques. These techniques typically produce irregularly sized pieces that are often larger than desired, inconsistent, and therefore difficult to integrate into the lesion properly. These techniques also tend to produce a great deal of pressure and/or heat, and can require many minutes to process, which can compromise a high proportion of the living cells. They can also take significant time allowing for degrading surface oxidation. Lab viability testing of these techniques has shown to compromise as much as 70-80% of the living cells, leaving only 20-30% viability. There are known bone mills that work adequately, but they tend to be used to process larger amounts of bone into morsels in the range of 3 mm diameter or larger. Some apply a great deal of heat pressure compromising the live cells. Others are bulky, motor driven, complicated, expensive devices that require disassembly and cleaning. Others still have large expensive disposable components that attach to large, complicated, motors.

Once the bone section is removed from the Patient, the very costly operating room procedure halts while the Paste is processed.

### SUMMARY

Described herein is a unique rotary cutting blade contained in a small handheld portable housing, the Device, that is used to transform a section of bone, articular cartilage and other associated Elements into a fine Paste with a minimum of cell viability loss. We describe a process to mill smaller amounts of Elements to produce a consistent morsel size under 2 mm to make a Paste that maintains a high proportion of living cells. This then produces a high level of regeneration in an articular cartilage lesion, or bone graft.

In an embodiment, a device is provided for slicing a cylinder-shaped plug of articular cartilage and bone into uniform particles includes a morselizer disc with a first side having at least one recessed area with an area of at least 100 square millimeters and a depth of at least 0.2 mm. An opening passes through the morselizer disc at an edge of the recessed area with an edge of the opening having a cutting blade with a cutting edge elevated above the recessed area; the cutting edge has multiple, adjacent, rounded concavities presenting a plurality of adjacent chutes and teeth along the cutting edge-these concavities are configured to cut off layers of bone and cartilage while simultaneously morselizing the layers of bone and cartilage into uniform particles. The morselizer disc is within a housing having a handle configured to be held in one hand; the cutting blade engages a drive shaft, the drive shaft configured to couple to a rotary drill. A hollow tube on the housing having inner diameter smaller than a width and a length of the recessed are of the morselizer disc guides of the plug of articular cartilage and bone into the recessed area of the morselizer disc. A plunger extending into the hollow tube is configured to press the sample of articular cartilage and bone into the recessed area, and a removable receptacle residing on the housing receives the morselized cartilage and bone.

In another embodiment the cutting blade has a straight edge without chutes and teeth. In another embodiment, there is no recess, and the cutting edge is elevated above a body of the morselizer disk by between 0.2 and 2 millimeters.

### BRIEF DESCRIPTION OF THE DRAWINGS

The accompanying drawings, which are included to provide a further understanding of the embodiments and are incorporated in and constitute a part of this specification, illustrate embodiments and together with the description serve to explain the principles of the illustrated embodiments.

FIG. 1 is a perspective view of one portable rotary bone and articular cartilage step morselizer Device from the front, in accordance with an embodiment.

FIG. 2 is a rear perspective view of the Device of FIG. 1.

FIG. 3 is a front view of the Device of FIG. 1.

FIG. 4 is a rear view of the Device of FIG. 1.

FIG. 5 is a side view of the Device of FIG. 1.

FIG. 6 is top view of the Device of FIG. 1.

FIG. 7 is bottom view. of the Device of FIG. 1

FIG. 8 is a perspective view of an embodiment of the Device of FIG. 1 with an Elements plug of bone and cartilage.

FIG. 9 is a side sectional view of the Device with Elements bone plug of FIG. 8.

FIG. 10 is a is an exploded perspective view of the Device with an Elements plug of bone and cartilage of FIG. 8.

FIG. 11 is a perspective view of a tabletop holder used to hold the detachable bone receptacle.

FIG. 12 is a perspective view of a detachable bone receptacle secured in the tabletop holder of FIG. 11.

FIG. 13 is a perspective view of a surgical drill rotating a shaft and rotary morselizer disc.

FIG. 14 is a side sectional view of the surgical drill rotating a rotary morselizer disc.

FIG. 15 is an enlarged top sectional view of the surgical drill rotating a circular step blade.

FIG. 16 is a front perspective view of one rotary morselizer disc.

FIG. 17 is a rear perspective view of the rotary morselizer disc of FIG. 16.

FIG. 18 is close-up perspective front view of a rotary morselizer disc with recess on the first surface and one of the blades.

FIG. 19 is close-up perspective rear view of one rotary morselizer disc showing one of the blades with conical cutouts and teeth.

FIG. 20 is an enlarged side sectional view of one rotary morselizer disc and blade with Element plug.

FIG. 21 is an enlarged side sectional view of one rotary morselizer disc and blade with Element plug.

FIG. 22 is an enlarged side sectional view of the rotary morselizer disc with morselized Elements.

FIG. 23 is an enlarged side sectional view of one morselizer disc blade demonstrating an angle of cutout and angle range of the blade at the point of the conical cutout.

FIG. 24 is an enlarged side sectional view of the morselizer disc blade demonstrating the angle of the cutout, the angle of the blade at the recess of the conical cutout, and the disc thickness.

FIG. 25 is an exploded view of an alternative configuration with a spring mechanism for pressing the plunger.

FIG. 26 is a front perspective view of an alternative configuration with a spring mechanism for pressing the plunger.

FIG. 27 is a sectional view of an alternative configuration with a spring mechanism for pressing the plunger and a surgical drill for rotating the morselizer disc.

FIG. 28 is a front perspective close-up view of an alternative morselizer disc with a straight blade.

FIG. 29 is a front perspective closeup view of an alternative rotary morselizer disc with a straight blade.

FIG. 30 is an enlarged side sectional view of the rotary morselizer disc blade and Element plug.

FIG. 31 is an enlarged side sectional view of the rotary morselizer disc with a blade that is flat.

FIG. 32 is a front perspective close-up view of an alternative morselizer disc with a straight, unrecessed, blade raised from the disc surface.

FIG. 33 is a front perspective close-up view of an alternative morselizer disc with a blade with conical cutouts that is raised from the disc surface.

FIG. 34 is a front perspective close-up view of an alternative morselizer disc blade with a blade with conical cutouts.

FIG. 35 is an enlarged side sectional view of the morselizer disc blade with an Element plug being sliced to give morselized elements.

FIG. 36 is an enlarged side sectional view of the morselizer disc blade with conical cutouts showing an Element plug being sliced to give morselized elements.

FIG. 37 is an enlarged side sectional view of the rotary morselizer disc blade with the Element plug tipping over.

FIG. 38 is a front perspective view of one Device coupled through a geared reduction box coupled to a high-speed dental drill.

FIG. 39 illustrates a flow chart of a process for operating one Device.

### DETAILED DESCRIPTION

In the following description of embodiments, numerous specific details are set forth in order to provide more thorough understanding. However, note that the embodiments may be practiced without one or more of these specific details. In other instances, well-known features have not been described in detail to avoid unnecessarily complicating the description.

Embodiments are described herein with reference to the figures wherein like reference numbers indicate identical or functionally similar elements.

Embodiments are intended for use in a sterile surgical operating room where a section of unprocessed Elements is removed from a Patient as a cylindrical plug or other similar shape and made into a fine Paste to be immediately reinserted into the body. The Device is suitable for the repair of articular cartilage in human or mammal Patients using articular cartilage Paste graft techniques, and also for orthopedic bone grafting procedures. The Device has very few parts which can be produced very economically and provided as a sterile disposable product, or as a reusable device.

The Device quickly produces bone Paste that experiences minimal pressure, heat, and exposure during processing through the device. The device utilizes a circular rotating morselizer disc that has one or more recesses that each end with a hole adjacent to a sharp blade. This produces a stepping and shaving action that occurs on bone and cartilage segments pushed onto the rotating cutting disc. Embodiments having multiple recesses and associated blades on the morselizer disk provide stepping and shaving occurrences for each recess, thus providing multiple stepping and shaving occurrences per revolution of the blade. The holes in the disc allow the morselized Elements to pass through the disc to be deposited into a removable receptacle. An embodiment having four recesses and four blades provides four shaves per disc revolution.

The rotary cutting blade is driven by a shaft attached to a typical sterile surgical electric variable speed drill. The shaft is inserted into a hole in the Device to engage with the rotary morselizer disc. A central rotating cutout in the blade engages with a matching shape on an end of the shaft, such as a square or star. The drill is then activated to slowly rotate the cutting disc as a plunger feeds the bone through the rotating cutting disc, thus macerating the Elements. When actuated, the drill rotates at approximately 100-500 revolutions per minute (RPM) thus shaving 400-2,000 thin layers per minute.

The recesses on the surface of the rotatory cutting disc are made to a depth of approximately 0.1 mm to 1 mm deep, or another depth to produce the desired shave section thickness as needed to produce the optimal Paste particle size. The blades in an embodiment have angled conical cutouts on the blade surface that result in teeth along the cutting edge which shave and simultaneously crack and separate the brittle bone Element layers into tiny morsels with a diameter of 2 millimeters or less.

The device housing incorporates a hollow tube opposing the cutting blades that an unmorselized, raw, Elements plug is placed into. A plunger, attached to the Device, presses the plug of bone and cartilage towards the rotary cutting blade. The ergonomic Device is held in one hand of an operator. The operator's other hand holds the surgical drill with a shaft that rotates the blade at a slow speed. As the disc rotates, the plunger is pressed by the thumb associated with the hand holding the device. The result is a coordination between the operator's hands to adjust rotation speed to a slow rotation, while applying the required appropriate pressure with the thumb or finger to morselize the Elements. Alternately, the plunger can be spring-activated without using the thumb.

The Element particles pass through the openings, or cutouts, in the rotating morselizer disc and drop into a removable receptacle until the bone section is fully shaved. The receptacle is then removed from the Device for inspection. The receptacle is affixed to the housing of the Device in a manner that it can be easily removed for inspection, mixing, and insertion of its contents into the trephine used to insert the Elements into the body. Once the receptacle is removed from the Device, it is securely placed onto a holding platform that can reside on a table for further processing if needed. The receptacle is of a clear material and has markings on it indicating the depth of the material it contains. Additives may be introduced in desired proportions by estimating the volume of morselized Elements in the receptacle. The paste is compacted, and the height is estimated, according to markings on the receptacle, then, the footprint volume of the receptacle is multiplied by the height of the Elements to establish the volume. The proper amount of measured additive is then mixed into the morselized elements in the receptacle before being introduced into a trephine that can be inserted into an opening on the receptacle for filling with a minimum of time and exposure. The trephine is removed and inserted into the body and the Elements are pressed into place by a plunger within the trephine.

An embodiment is a handheld rotary bone and articular cartilage rotary step morselizer disc and housing, the Device, used to transform a section of bone, articular cartilage and other associated Element plug into a fine Paste with a maximum of cell viability maintained. The relatively-small hand-held device contains a rotary morselizer disc that contains blades to morselize the articular cartilage and bone Element plug into consistently sized particles in a few seconds in a sterile surgical operating theater. Features of each blade on the morselizer disc interact with the Element plug in a single step without contact with other parts of the Device.

In embodiments, the morselizer disc is fabricated from hardened steel, stainless steel, hardened stainless steel, titanium, or ceramic.

The morselizer disc has a first surface facing towards the Element plug, and a second surface facing away from the Element plug of cartilage, bone, and sometimes marrow. The Element plug is placed inside a tube on the Device that securely holds it as a plunger presses it towards the blade. The disc has at least one recess on the first disc surface that allows the Element plug to precisely move to a predetermined depth where they then meet a sharp angled blade that slices off the Element plug at a predetermined thickness. There is an elongated hole through the disc extending from the angled blade surface that allow the Elements to pass through the disc as it rotates. Multiples of the recesses, apertures and blades can be arranged in a circular pattern on the disc, four being used in a particular embodiment.

In an embodiment, the angled surface of the blade facing the second surface has a plurality of conical shaped cutouts that create chutes and cause the leading edge of the blades to have teeth. As the blade shaves off a segment of the brittle Element plug, the teeth and conical cutouts differentially lift, crack, and separate that segment, resulting in uniform pieces according to the size of the conical cutouts and teeth. The smaller the conical cutouts and teeth, the smaller the particles. This, combined with depth of the recesses, determines overall size of the morselized elements.

The device in an embodiment creates a fine paste of elements broken down into 0.2 mm to 1 mm pieces. By way of example, if the recess is 0.2 mm deep, the resulting shaved sheet would be 0.2 mm thick. If the conical cutouts have diameter of 1 mm, and teeth are spaced 2 mm apart, the resulting differential lifting of the slice from the Element plug varies from approximately 0.25 to 0.5 wide. This would result in particles consistently 0.2 mm thick by 0.25 mm to 0.5 mm in size. This cutting procedure does so with a single pass of a single blade without any other interaction of other parts of the device.

In an embodiment, the rotary morselizer disc is housed inside a plastic or metal housing. The housing has a first and second side matching the disc sides. The second side has an ergonomic handle attached to that is held in one hand. It has an oval hole matching the blades on the rotating disc that eject the Elements towards a receptacle. It also has a recess that engages the removable receptacle. A first side housing engages into the second side by mean of a turn-and-lock mechanism to disassemble for a reusable device, or the two sides can be permanently attached by a click interface, sonic welding, or the like for a one-time-use disposable device. The first side has a hollow tube that extends forward aligned with the blades in the rotary morselizer disc to receive the morselized Elements. A second tube is centrally located to receive a rotating drive shaft to rotate the disc.

In an embodiment, the Elements inside the hollow tube are pushed towards the rotating cutting disc by a plunger operated by the thumb of the hand holding the Device as the other fingers of that hand grip the handle. The plunger has an extended arm with a hole in it that the rotating shaft to passes through. This unitizes the device and aligns the plunger to the hollow tube for easy insertion.

In an embodiment, the rotating shaft is placed into the chuck of a sterile rotary surgical or other drill and the plunger is slid onto the shaft. The device and drill can reside on a sterile table beside an operating table, awaiting use. Once the device, held in one hand, is loaded with Elements, the other hand of the operator holds the drill, and the shaft is inserted into the central tube engaging the rotary morselizer disc. The thumb of the hand holding the device depresses the plunger as the other hand operates the drill at a slow speed. The operator will develop a sense of a combination of speed and adequate thumb pressure applied to achieve proper maceration of the Elements while maintaining the least amount of speed and pressure. A key element of the device is to morselize the Elements with the least amount possible of frictional heat, typically maintaining the morselized elements near room temperature, and the least amount of compression pressure. The slow rotation speed, and moderate thumb pressure, serve to apply the least heat and compaction pressure, so as to damage as few live Element cells as possible.

The removable clear plastic receptacle catches the morselized Elements. It is attached to the second side of the housing by a friction or click fit. The receptacle is loaded with morselized Elements from one or more plugs of the Elements in succession using the Device. The receptacle is then removed and placed onto a secure holding platform that resides on a table. The receptacle is stabilized, and the contents are inspected and further processed as needed. The Elements can also be compacted for measuring, using the plunger. The receptacle has indicator lines along the side to estimate a height of the compacted elements. A formula multiplying the footprint volume of the receptacle times the height of the Elements can be used to estimate the volume of Elements in the receptacle. This can be useful when introducing additives that need to be measured and added to a predetermined amount of Elements. As an example, 1 mm of element multiplied by the area of the footprint of the receptacle could be 0.12 cc. 5 mm height of element would equal 0.6 cc. A 10% additive could be calculated and introduced of 0.06 cc. A small spatula is used to mix the two.

A hollow tube is incorporated on the receptacle that tightly fits a hollow toothless trocar. The spatula is then used to push the contents of the receptacle into the trocar for introduction into the body of the patient. It should be noted that, due to the simple operation of the device, the entire process can be completed in a few minutes thereby maintaining the highest level of cell viability and saving valuable operating time.

The rotary morselizer disc can have other blade configurations as detailed in the illustrations to achieve a similar result.

Persons skilled in the art will readily appreciate that various aspects of the present disclosure can be realized by any number of methods and articles configured to perform the intended functions. Stated differently, other methods and articles can be incorporated herein to perform the intended functions. It should also be noted that the accompanying drawing figures referred to herein are not all drawn to scale but may be exaggerated to illustrate various aspects of the present disclosure, and in that regard, the drawing figures should not be construed as limiting. Finally, although the present disclosure may be described in connection with various principles and beliefs, the present disclosure should not be bound by any theory.

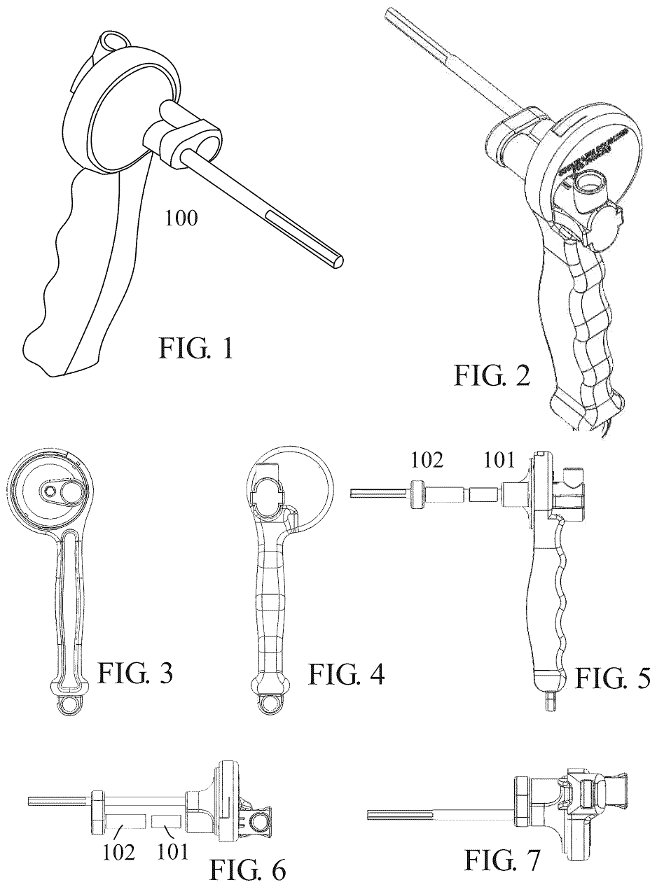

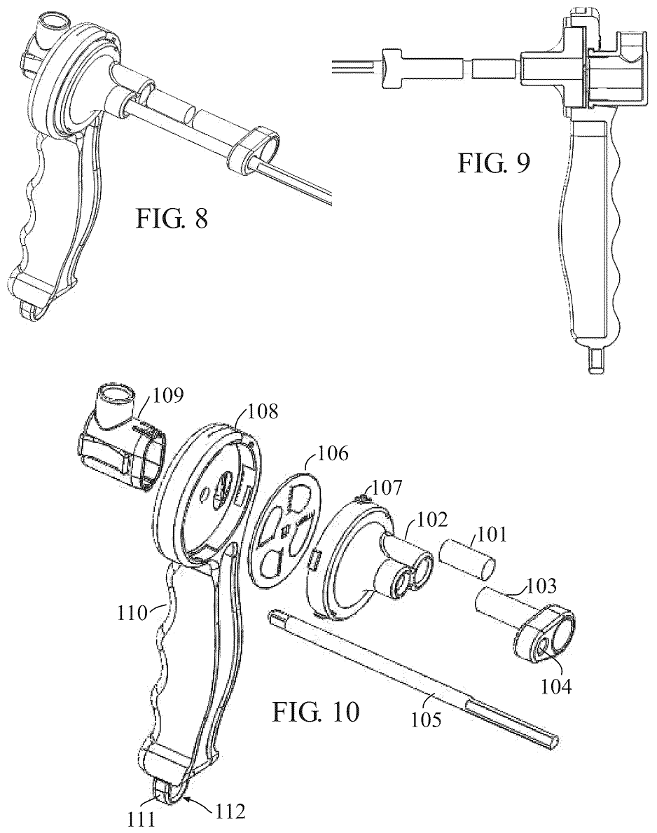

FIGS. 1 and 2 depict perspective views of the embodiment of the device 100, in the orientation it is typically used. FIGS. 3 and 4 depict views from the front and back. FIGS. 5 and 6 depict side and top, views of the device 100, with the articular cartilage and bone Element plug 101, about to be inserted into the hollow tube 102, and the plunger 103, about to push the plug in towards the rotary morselizer disc. FIG. 7 depicts the plunger 103, fully depressed into the device 100. FIG. 8 depicts a perspective view in the same configuration as FIGS. 5 and 6. FIG. 9 depicts a cut-away side view of the device in the same configuration as FIG. 8.

FIG. 10 depicts an exploded perspective view of the device. The plunger 103, that pushes the Element plug 101, into the hollow tube 102, the hollow tube having inner diameter smaller than a width and a length of the recessed area of the morselizer disc. The plunger 103, includes an extended arm with a circular hole 104, that engages the drive shaft 105. The drive shaft rotates the rotary morselizer disc 106, housed within the device front housing 107, and rear housing 108. The Elements, once morselized, fall into the receptacle 109. A handle 110 extends down from the rear housing, 108 with finger grip areas 110, to be securely held by the palm and fingers of one hand, leaving the thumb free to activate the plunger by depressing the plunger 103. The front housing 107, engages into the back housing 108, by means of a twist-and-lock mechanism for disassembly, or the housings can be permanently attached by a click interference element, sonic welding, or the like. The circular rotary disc 106, with four blade elements (the number may vary), resides inside the housings and is free to rotate due to a loose fit. The rotating shaft 105 engages into the rotary disc by passing through the central hole in the front housing. The plunger 103, slides onto the rotary shaft 105 attached through a hole 104. There is a protrusion on the bottom of the handle on the back housing 111. It has a round hole in it 112, for the purpose of testing the size of the articular cartilage and bone Element plug 102. If it passes through the hole 112, then it will fit into the tube 102, on the front housing 107. If it does not, then it needs to be trimmed down so it will pass through the tube 102, freely.

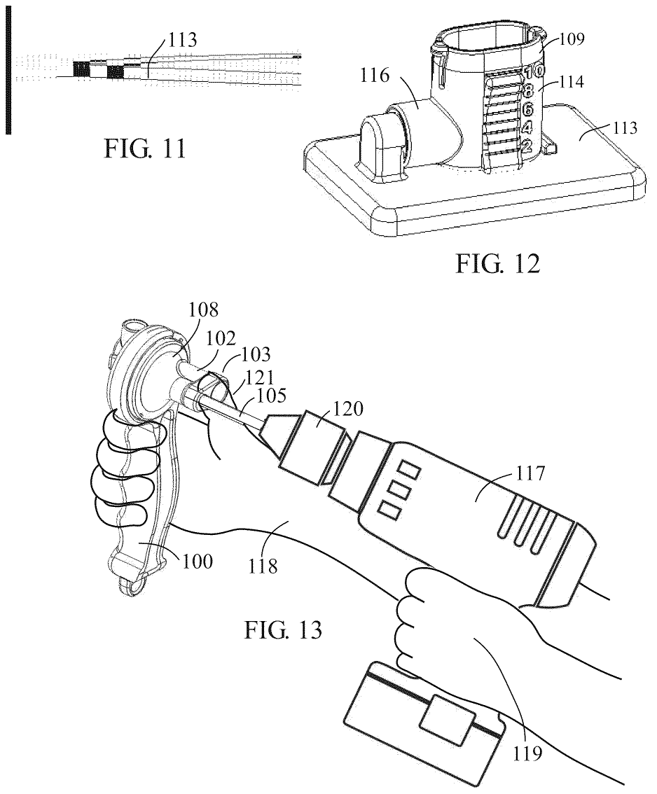

FIG. 11 depicts a desktop holder, in a position to receive the receptacle 109.

FIG. 12 depicts the receptacle, once it is filled, rotated 90 degrees. It is then secured in the tabletop holder 113. In this position, the contents can be examined, manipulated, and mixed with other additives. The morselized Elements, once in the receptacle 109, can be compacted using the plunger 103, so that the volume can be approximately measured using the indicator lines 114, on the side of the receptacle 109. A formula can be used to determine the volume of the Elements by multiplying the height of the Elements times the known volume of the interior foot print of the receptacle 109. Knowing the volume of the Elements can be useful for determining the volume of any additives needing to be added and mixed in. A hollow trocar, typically used to insert Elements into the body during non-invasive surgery can be inserted into the hollow tube 116, on the receptacle 109. Then the Elements can be pushed into the trocar using a small spatula, for insertion into the body. The receptacle 109, and desktop stand 113, are designed so that the Elements can be efficiently processed with minimal exposure, contamination, and waste in the shortest amount of time.

FIG. 13 depicts the device 100, being held in the right hand 118, while a rotary drill 117, is held in the left hand 119. The rotary shaft 105, is attached to the drill chuck 120, and inserted into the front housing 108, engaging with the rotary morselizer disc. The hollow tube 102, is filled with the Element plug and the plunger 103, is inserted into the hollow tube 102. The fingers and hand 118, of the right hand 118, holds the handle 110, so the thumb 121, is free to push on the plunger 103, forcing the Element plug towards the rotary morselizer disc. The slowest possible drill rotation speed, combined with the lowest required thumb pressure, morselizes the elements with the lowest combination of frictional heat and pressure, thereby preserving the highest degree of live Element cells during the maceration process.

All of the components of the reusable device can withstand temperatures of over 135 degrees Celsius (275F), so they are appropriate for typical autoclave steam sterilization. A surgical drill, commonly used in sterile operating room surgery, can be sterilized in an autoclave along with the device. Alternately, the Device can be supplied as a onetime use disposable product.

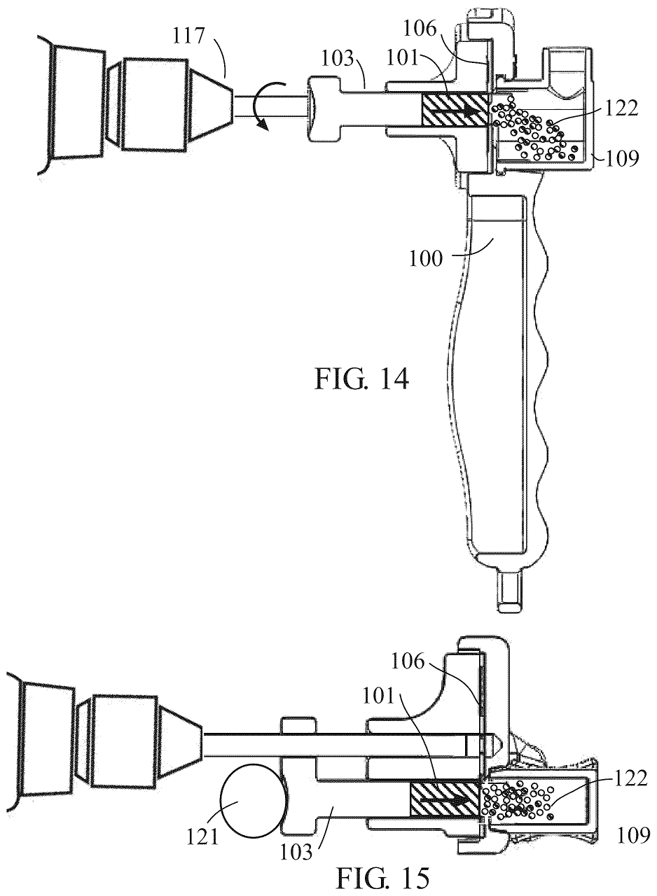

FIGS. 14 and 15 depict sectional views of the device 100, FIG. 14 is a side view and 15 is a top view. The drill 117, rotates while the thumb 121, depresses the plunger 103. The Element plug 101 is pushed towards the rotary morselizer disc 106 and is morselized by the conical cutout toothed blades. The morselized Elements 122, pass through the rotary morselizer disc 106, and are deposited into the receptacle 109.

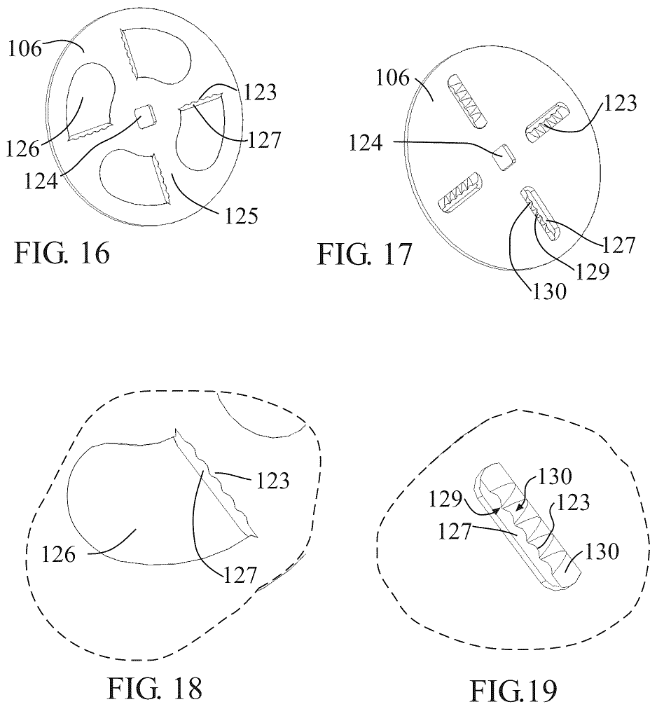

FIGS. 16 and 17 depict the rotary morselizer disc 106, with four blades 123, dispersed radially, although the number of blades may vary. The centrally located aperture 124, is a square although other shapes may be used that engage with the metal rotary shaft. FIG. 16 depicts the front surface 125, that engages with the Element plug, which has four recessed areas 126, corresponding to each blade. Between the blades 123, and the recesses 126, there are apertures 127, through which the Elements pass as they are sliced and morselized. FIG. 17 depicts the rear surface of the disc, which is the side away from the Element plug where. The apertures 127 or openings, adjacent conical cutouts 129 or chutes, also known herein as concavities, and blade teeth 130, can be seen. FIG. 18 depicts a closeup view of the disc front side 125, with a recess 126, an aperture 127, and the conical cutouts with the toothed cutting edge 123. FIG. 19 depicts the disc back side with the angled conical cutouts surface. The conical cutouts 130, are cut into the angled blade surface 130, at a greater angle, so as to create chutes and protruding teeth 129, in between them. The adjacent concavities have radius from 1-3 mm. The cutting edge is elevated above the recessed area of the morselizer disc by between 0.2 and 2 mm, and in embodiments the cutting edge is level with a surface of the morselizer disc.

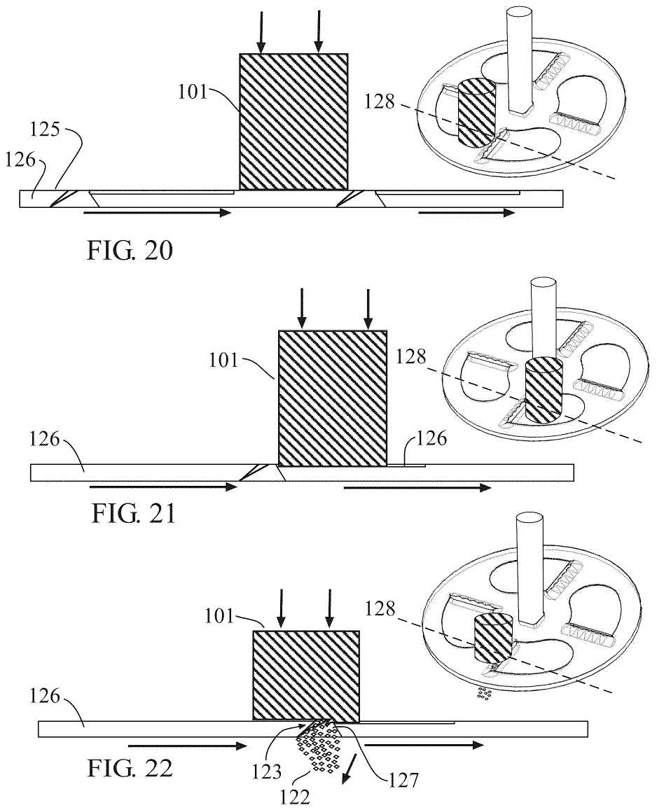

FIGS. 20, 21 and 22 depict a side sectional view of the rotary morselizer disc 106, without other components, but with an articular cartilage and bone Element plug 101, shown. The dashed line 128, depicts the location of the sectional view cutting plane shown in FIGS. 20, 21, and 22. The plug 101, remains stationary as the disc 106, rotates in all three figures. FIG. 20 depicts the disc 126, in one rotational position, with the Element plug 101, residing on the disc first surface 125. FIG. 21 depicts the disc 126, in second rotational position, with the Element plug 101, dropping into the recess 126. FIG. 22 depicts the disc 126, in a third rotational position, with the Element plug 101, being cut and morselized by the blade and conical cutouts 123. The morselized Elements 122, pass through the aperture 127, to be deposited into the receptacle which is not shown. The action of the plug dropping into the recessed area so that it meets the conical cutouts cutting blades at a precise depth is a critical component of the device design. It precisely establishes the thickness of the cut, time after time as the disc rotates and the plug is pushed into each recess.

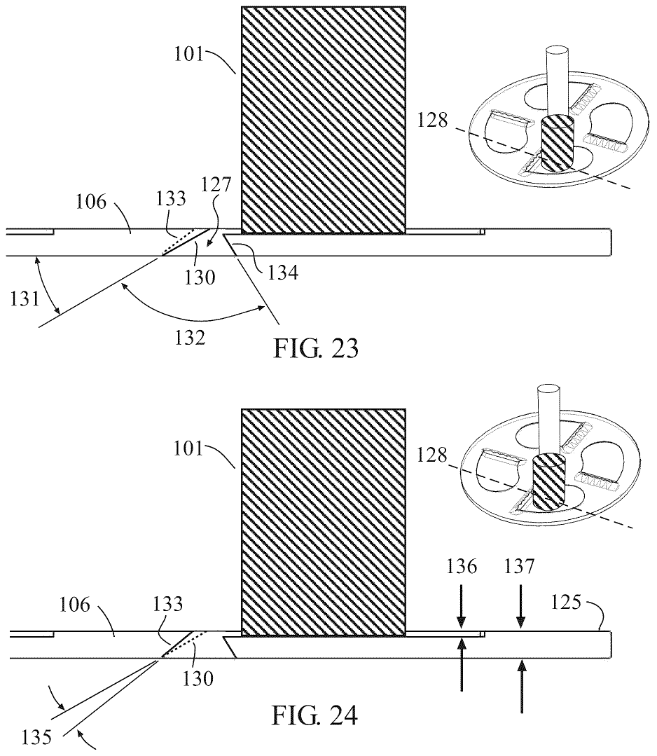

FIG. 23 depicts a sectional view of the rotary morselizer disc 106, without other components, but with one articular cartilage and bone Element plug 101, shown. The dashed line 128, depicts the location of the sectional view cutting plane shown in FIGS. 23 and 24. The angle range 131, shows the angled cutting surface 130, (black line), in relation to the front and rear surface of the disc. The optimal angle 131, is 30 degrees plus or minus 10 degrees. The angle 132, depicts the angle between the angled cutting blade 130, and the opposing angled surface 134, that pushes the Elements through the aperture 127 of the disc. This optimal angle is 90 from angle 131, as produced by the cutting mill end used to make the feature.

FIG. 24 is a sectional view of the rotary morselizer disc 106, without other components, but with one articular cartilage and bone Element plug 101, shown. The angle range 135, shows the conical cutout angle, (black line), in relation to the angled cutting surface 130, (dashed line). The optimal angle range 135, is 5 degrees plus or minus 2 degrees. On the right of FIG. 23, arrows 136, depict the depth range of the recesses from the front surface of the rotary morselizer disc, which ranges from 0.1 to 2 mm. The thickness of the cutting blade 137, can be in a range from 0.5 to 3 mm.

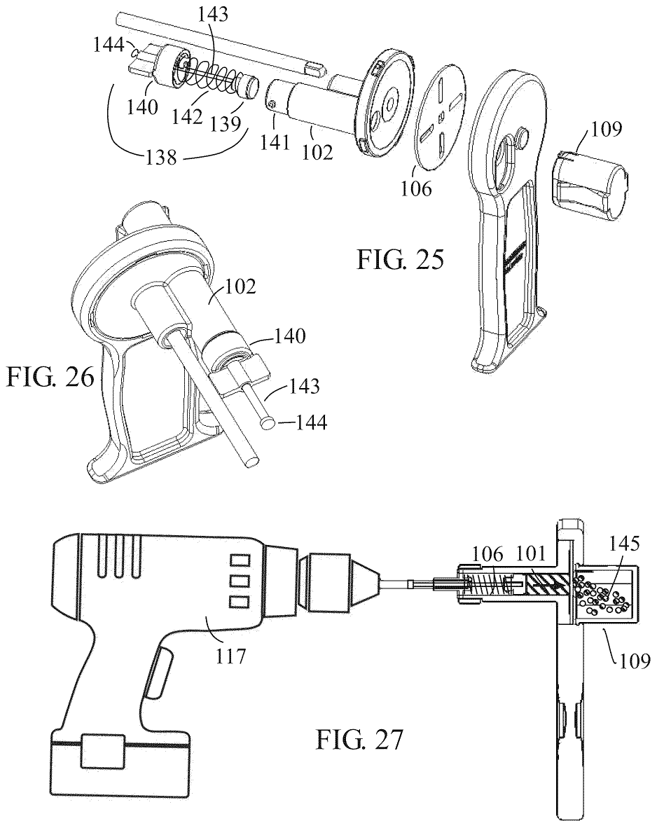

FIG. 25 depicts the device in an exploded view showing an alternative feature for the plunger to push the Elements towards the rotary morselizer disc 106. It incorporates a spring-loaded plunger assembly 138, in place of the thumb operated plunger 103. Once the Element plug (not shown) is loaded into the hollow tube 102, a spring plunger tip 139, is inserted and the cap 140, on the opposite end is pushed to engage with the hollow tube end 141 by a turn-lock feature, thereby compressing the spring 142, to push the Element plug towards the morselizer disc 106. A metal rod 143, running through the spring assembly 138, unitizes the parts and stops the plunger tip 139, just as it reaches the rotary morselizer disc 106, by means of a stop on the rod end 144. FIG. 26 depicts the device assembled with Elements (not shown) in the hollow tube 102, and the cap, 140 engaged onto it. The metal rod is pushed out due to the Element plug 101, compressing the spring 142.

FIG. 27 depicts a rotary drill 117, used to rotate the rotary morselizer disc 106, and morselize the Elements 101, as previously described. The Spring mechanism 138, can be seen pushing the Element plug 101, as the rotary morselizer disc 106, morselizes the Element plug 145, and passes into the receptacle 109.

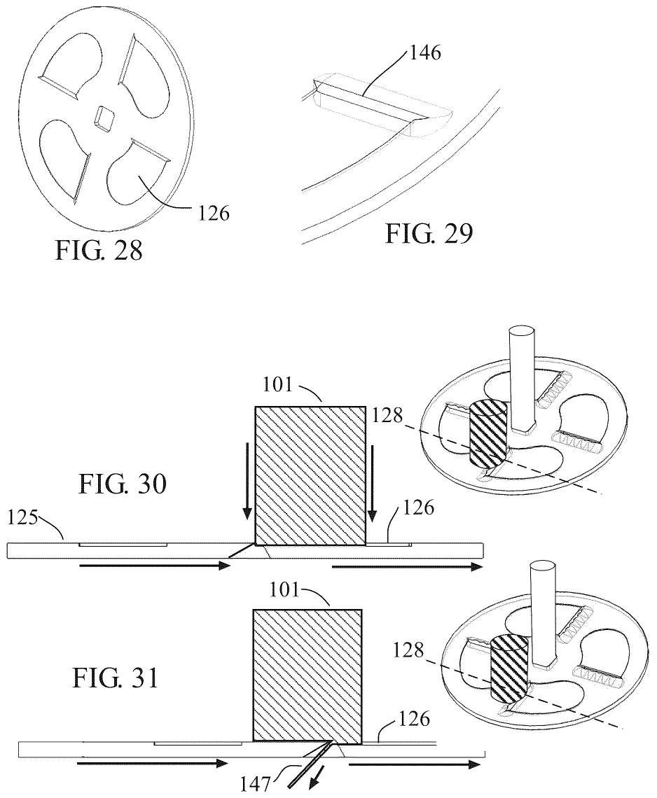

FIG. 28 depicts a perspective view of an alternative construction of the rotary morselizer disc that has the recesses 126, on the first surface, but does not have the conical cutout cutouts in the blade. FIG. 28 is a perspective close-up view that shows the straight edge of the blade 146, on the angled surface, without conical cutouts.

FIG. 30 depicts a sectional view of the rotary morselizer disc 106, without other components, but with one articular cartilage and bone Element plug 101, shown. The dashed line 128, depicts the location of the sectional view cutting plane. The bone plug is pressed to the recess 126, on the disc surface 125.

FIG. 31 depicts a sectional view of the rotary morselizer disc, 106, in one position with the element plug 101, on the recessed surface 126, being sliced by the blade 146, but not morselized. However, due to the very thin slice across the Elements, the sheet is easily broken into small segments by mixing and mashing once it is in the receptacle.

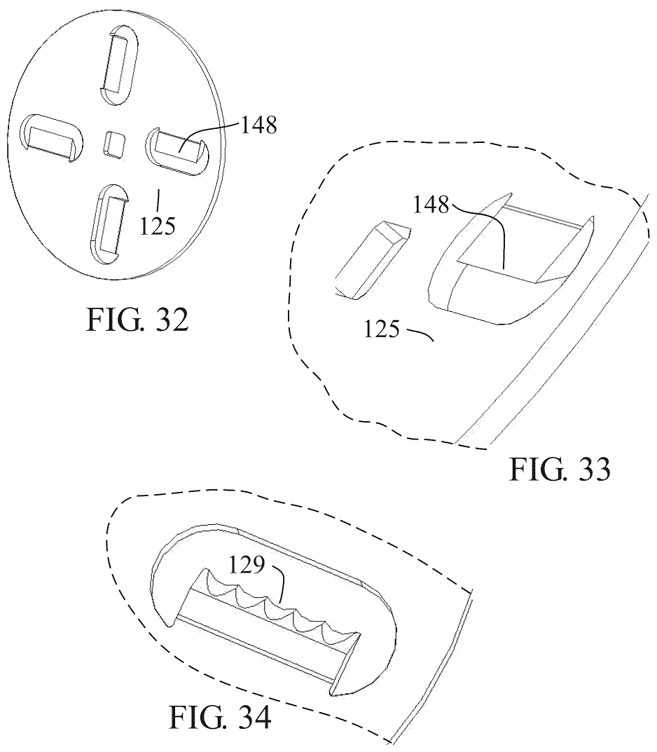

FIG. 32 depicts yet another alternate construction of the rotary morselizer disc 106. There are no recessed sections 126, on the first surface of the disc as previously described. Alternately, the blade edges 148 rise above the first surface of the disc 125. This is achieved by machining or by bending the blades after they are machined.

FIG. 33 depicts a perspective close up view of the raised straight blade 148.

FIG. 34 depicts the same raised blade construction with conical cutout cutouts and teeth 129, previously described, that morselize the Elements.

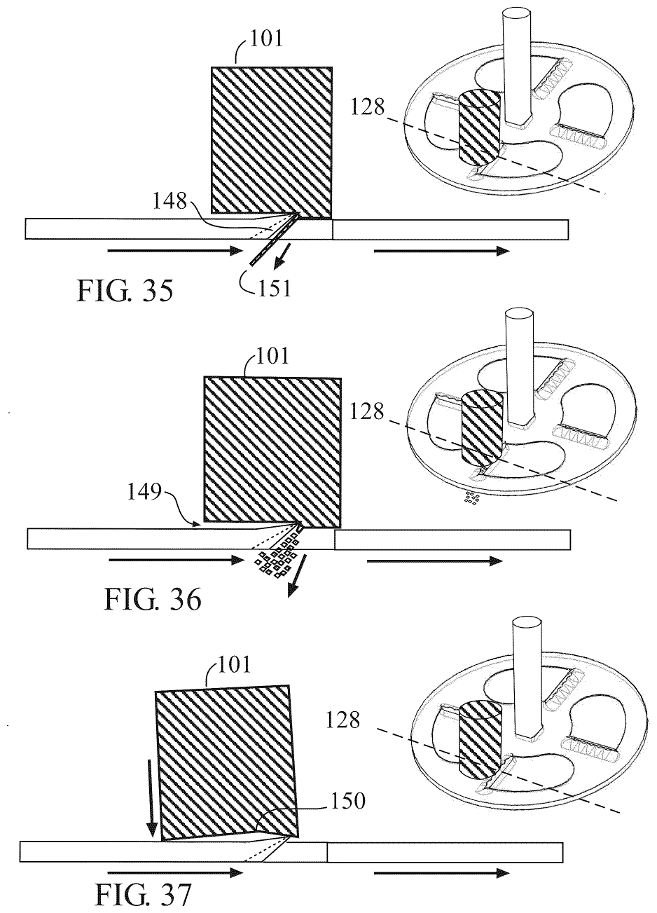

FIG. 35 depicts a sectional view of the straight raised blade 148, in one position with the element plug 101. The dashed line 128, depicts the location of the sectional view cutting plane. The raised blade 148, is slicing off a sheet of Element. Note the gap resulting in the portion of Element that has the sheet cut away 149.

FIG. 36 depicts a sectional view of the straight raised blade 148. The dashed line 128, depicts the location of the sectional view cutting plane. The raised blade 148, is slicing off a sheet of Element as the conical cutout cutouts 129, previously detailed, morselize the Elements. Note the gap resulting in the portion of Element that has the sheet cut away 149.

FIG. 37 depicts a sectional view of the straight raised blade 148. The dashed line 128, depicts the location of the sectional view cutting plane. Just as the raised cutting blade 148, leaves the element plug 101, and the plug tips over. There is an uneven surface 150, resulting on the bottom of the plug 101. This uneven surface, when the next blade approaches, causes more uneven cutting. The result is that, although the Elements are cut, there are gaps in the cuts and the cutting becomes uneven on the bottom of the plug which is less efficient as the blades continue to pass, nipping smaller irregular portions. Experiments showed that although this design was viable and an alternative, a rotary morselizer disc with recesses, that kept the Element plug flat on the bottom, along with the conical cutouts on the blade, resulted in much more efficient cutting, and more uniform morselizing. Therefore, the disc with recesses and blade with conical cutouts are used in embodiments.

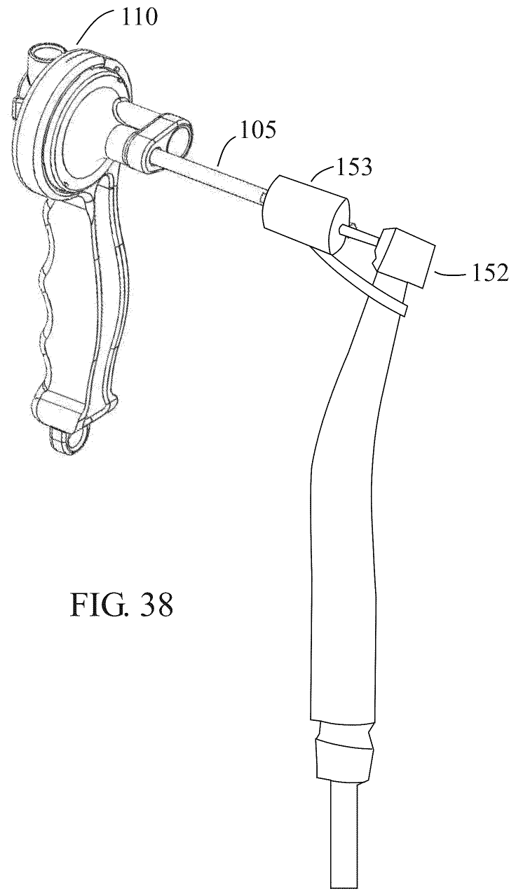

FIG. 38 depicts a perspective view of the device paired with a high-speed dental drill 151. In order to rotate the drive shaft 105, and rotary morselizer disc 106 (not shown), at a slow speed of under 500 rpm, with a dental drill that rotates at thousands of RPM, an interface 152, between the drive shaft 105, and the dental drill 151, is attached that has a gear reduction mechanism inside it so as to operate the Device at the proper speed of under 500 RPM.

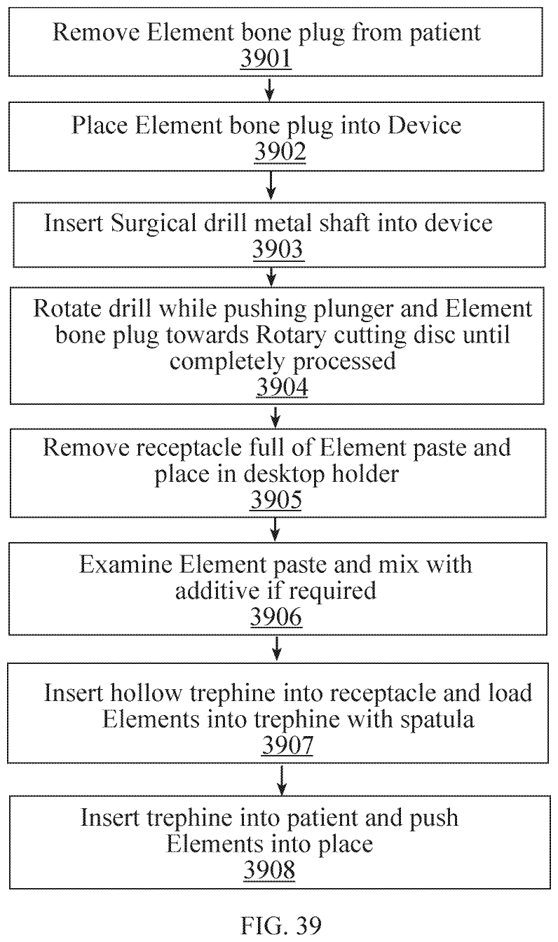

FIG. 39 depicts a flow chart of a process for operating the Device. A plug of Elements, which may include bone, cartilage, and/or marrow, is removed from the patient, the plug is inserted into the Device. The shaft, if not already present, is inserted into the Device to engage the morselizer disc, and the plunger is inserted into the device. An operator then causes the drill to rotate the morselizer disc until sufficient of the plug is processed. The receptacle is then removed, contents of the receptacle are inspected and any additives desired for implantation such as a gel and/or growth factors are added and blended in, and the contents, now a fully processed paste, are transferred into a trephine. The trephine is then used to implant the paste into a defect in bone or cartilage of the patient.

### EXAMPLE

Reference will now be made in detail to an embodiment, an example of which is illustrated in the accompanying drawings. Without intending to limit the scope of the disclosure, the following example illustrates how various embodiments of the Device may be made and/or used. The language used in the specification has been principally selected for readability and instructional purposes, and it may not have been selected to delineate or circumscribe the inventive subject matter. It is therefore intended that the scope of the disclosure be limited not by this detailed description, but rather by any claims that issue on an application based hereon. Accordingly, the disclosure of the embodiments is intended to be illustrative, but not limiting, of the scope of the disclosure, which is set forth in the following claims.

This example illustrates the use of an embodiment of the bone and cartilage step blade morselizer Device in a sterile operating room, by a single surgeon, to perform an articular cartilage paste graft in the knee of a Patient with an articular cartilage lesion on the bearing surface of tibia. It is typically performed with arthroscopic surgery as illustrated in FIG. 39. The surgeon prepares the articular cartilage lesion by trimming and debridement, and then the underlying bone is punched with small holes intended to receive the bone Paste.

The sterile disposable step slicer Device is removed from the sterile packaging and the included metal rotary shaft is inserted into a sterile surgical drill, then the plunger is slid onto the drive shaft and the assembly is set aside. The Paste receptacle is attached to the body of the Device to receive the Element Paste. The surgeon inserts a toothed hollow tube trephine into the Patient, bypassing the patella and inserts it into the intercondylar non weight bearing notch of the femur. The trephine is rotated or pounded to cut and remove 3901 a plug of bone and articular cartilage Elements that is approximately 8 mm×15 mm. The bone plug is then inserted 3902 into to the hollow tube on the Device. The rotary drive shaft on the drill is inserted 3903 into the device. The plunger is inserted into the hollow tube. The surgeon holds the Device in one hand and the drill in the other. he drill is activated to rotate 3904 the morselizer disc at a slow RPM of approximately 100-500. The thumb of the hand holding the Device depresses the plunger as the drill rotates the rotary morselizer disc. In less than 60 seconds, the blades slice and morselize the bone Element plug into the receptacle. Multiple introductions of Element plugs can be morselized until the desired amount is produced or the receptacle is full. The drill and shaft are removed from the Device.

The surgeon taps the Device onto a hard surface in a manner to force most of the bone into the receptacle and the receptacle is removed 3905 from the Device. The Receptacle is then placed in a holder on a table so the Paste can be inspected 3906, mixed with additives such as a gel if required, stirred, and further morselized if needed. The Paste can also be measured by compacting it into the receptacle using the plunger so it can be measured for volume. It can also have a volume of additive, such as a gel, introduced that is measured and introduced by a percentage calculation to the Paste. The Paste is then inserted 3907 into a toothless hollow tube trephine with a rod plunger inside. The trephine is inserted into the Patient and the morselized Elements are pressed 3908 into the prepared articular cartilage lesion. A hammer can be used to gently impact the Element Paste into the lesion. The process may be repeated if the lesion is large and requires more bone Paste. The process is then completed, and the Patient is prepped to completion. Within 6 weeks, new cartilage is formed healing the lesion.

A bone Paste processor that can be made of a few small plastic and metal components that are inexpensive to manufacture would lend itself to being disposable is desirable. Alternately, a simple small reusable Device that can be sterilized and used repeatedly could be beneficial. All of these desired features will be identified and explained in the detailed description.

What is claimed is:

1. A device for slicing a plug of articular cartilage and bone into uniform particles, comprising:
a morselizer disc with a first side having at least one recessed area with an area of at least 100 square millimeters and a depth of at least 0.2 mm;
the morselizer disc has an opening passing through the morselizer disc at an edge of the recessed area with an edge of the opening comprising a cutting blade having a cutting edge elevated above the recessed area;
the cutting edge has a plurality of adjacent rounded concavities presenting a plurality of adjacent chutes and teeth along the cutting edge configured to cut off layers of bone and cartilage while simultaneously morselizing the layers of bone and cartilage into uniform particles;
the morselizer disc disposed within a housing having a handle configured to be held in one hand;
the morselizer disc engaging a drive shaft, the drive shaft being configured to couple to a rotary drill;
a hollow tube extending on the housing has inner diameter smaller than a width and a length of the recessed area of the morselizer disc and configured to guide the plug of articular cartilage and bone into the recessed area of the morselizer disc;
a plunger extending into the hollow tube is configured to press the plug of articular cartilage and bone into the recessed area;
a removable receptacle residing on the housing to receive the morselized cartilage and bone.

2. The device of claim 1, the plunger being configured for actuation with a thumb of the hand holding the handle.

3. A device of claim 2, the removable receptacle being disposed on a second side of the morselizer disc.

4. A device of claim 1, the morselizer disc having a plurality of recesses each having a cutting edge.

5. A device of claim 1, a depth of the recessed area on the morselizer disc being less than 2 mm.

6. A device of claim 1, an angle of the cutting edge relative to a bottom of the recessed area of the morselizer disc being 30 degrees plus or minus 10 degrees.

7. A device of claim 6, there being from 3 to 10 adjacent concavities along the cutting edge, and a radius of the concavities is from 1-3 mm.

8. A device of claim 1, an angle of the plurality of adjacent rounded concavities of the cutting edge relative to an angle of the cutting edge being between 3 and 7 degrees.

9. A device of claim 1 the morselizing disc comprising steel, hardened steel, stainless steel, hardened stainless steel, titanium, or ceramic.

10. A device of claim 1, the morselizing disc having a diameter of from 25 mm to 60 mm and a thickness of from 0.5 mm to 3 mm.

11. A device of claim 1, wherein the plunger has an extending arm with a hole in it to engage with the rotary shaft.

12. A device of claim 1, the plunger being activated by pressure from a spring.

13. A device of claim 1, wherein the drive shaft is coupled to the morselizer disc through a geared reduction box.

14. A device of claim 1 sterilized and packaged in a sterile packaging for one-time disposable use.

15. The device of claim 1 that can be disassembled after use, cleaned, and sterilized for reuse.

16. A device of claim 2, the receptacle having indicator markings that establish a volume of morselized articular cartilage and bone in the receptacle.

17. A device of claim 2, the receptacle having a hollow tube extending from it configured to receive a hollow surgical trocar.

18. A method comprising:
providing the device of claim 2;
obtaining the plug of bone and cartilage from a patient;
placing the plug of bone and cartilage in the hollow tube of the device;
inserting the plunger into the hollow tube and applying pressure to the plunger; and
rotating the shaft to rotate the morselizer disc to morselize the plug of bone and cartilage.

19. A method of claim 18, the morselizer disc being rotated at a speed of from 100 to 500 revolutions per minute.

20. The method of claim 19 further comprising blending the morselized plug of bone and cartilage with a gel.

21. The method of claim 19 further comprising inserting the morselized plug of bone and cartilage into a surgical trocar and implanting the morselized plug of bone and cartilage in the patient.

22. A device for slicing a plug of articular cartilage and bone into uniform particles, comprising:
a morselizer disc with a first side having at least one recessed area with an area of at least 100 square millimeters and a depth of at least 0.2 mm;
an opening passing through the morselizer disc at an edge of the recessed area with an edge of the opening comprising a cutting blade having a cutting edge elevated above the recessed area;
the cutting edge configured to cut off layers of bone and cartilage while simultaneously morselizing the layers of bone and cartilage into uniform particles;
the morselizer disc disposed within a housing having a handle configured to be held in one hand;
the morselizer disc coupled to a drive shaft configured to couple to a rotary drill;
a hollow tube extending on the housing having an inner diameter smaller than a width and a length of the recessed area of the morselizer disc and configured to guide the plug of articular cartilage and bone into the recessed area of the morselizer disc;
a plunger extending into the hollow tube configured to press the plug of articular cartilage and bone into the recessed area;
a removable receptacle residing on the housing to receive morselized cartilage and bone.

23. A device for slicing a plug of articular cartilage and bone into uniform particles, comprising:
a morselizer disc having an opening;
an opening passing through the morselizer disc with an edge of the opening comprising a cutting blade having a cutting edge elevated above a body of the morselizer disc;
the cutting edge configured to cut off layers of bone and cartilage while simultaneously morselizing the layers of bone and cartilage into uniform particles;
the morselizer disc disposed within a housing having a handle configured to be held in one hand;
the morselizer disc coupled to a drive shaft configured to couple to a rotary drill;
a hollow tube extending on the housing and configured to guide the plug of articular cartilage and bone onto the morselizer disc;
a plunger extending into the hollow tube configured to press the plug of articular cartilage and bone onto the morselizer disc;
a removable receptacle residing on the housing to receive morselized cartilage and bone.
